SEQUENCE LISTING (23 sequences)

SEQ ID NO: 1 - DNA, 20 bp, Artificial sequence
  Single strand synthetic DNA oligonucleotide
agctggctggtggcagatgg

SEQ ID NO: 2 - DNA, 20 bp, Artificial sequence
  Single strand synthetic DNA oligonucleotide
cgttgatgccggtgaacgtg

SEQ ID NO: 3 - DNA, 25 bp, Artificial sequence
  Single strand synthetic DNA oligonucleotide
tagcctctttggggttgttctactg

SEQ ID NO: 4 - DNA, 25 bp, Artificial sequence
  Single strand synthetic DNA oligonucleotide
tggggcttcgtttagtctctacttc

SEQ ID NO: 5 - DNA, 25 bp, Artificial sequence
  Single strand synthetic DNA oligonucleotide
tagcctctttggggttgttctactg

SEQ ID NO: 6 - DNA, 25 bp, Artificial sequence
  Single strand synthetic DNA oligonucleotide
tagcctctttggggttgttctactg

SEQ ID NO: 7 - DNA, 36 bp, Artificial sequence
  Single strand synthetic DNA oligonucleotide
cgcggtcgacttatcaagaggtagaagagctgtaac

SEQ ID NO: 8 - DNA, 24 bp, Artificial sequence
  Single strand synthetic DNA oligonucleotide
gcgaacacgccgtgtttcaagggc

SEQ ID NO: 9 - DNA, 22 bp, Artificial sequence
  Single strand synthetic DNA oligonucleotide
ggaaacaagctggtagctgaca

SEQ ID NO: 10 - DNA, 21 bp, Artificial sequence
  Single strand synthetic DNA oligonucleotide
cctgggtcaacacaagatgct

SEQ ID NO: 11 - DNA, 20 bp, Artificial sequence
  Single strand synthetic DNA oligonucleotide
agcctcctctaccgccagaa

SEQ ID NO: 12 - DNA, 20 bp, Artificial sequence
  Single strand synthetic DNA oligonucleotide
gtgccagactcctccttgct

SEQ ID NO: 13 - DNA, 21 bp, Artificial sequence
  Single strand synthetic DNA oligonucleotide
ttaacttcctcacttgatcat

SEQ ID NO: 14 - DNA, 16 bp, Artificial sequence
  Single strand synthetic DNA oligonucleotide
accagaaccgagcaaa

SEQ ID NO: 15 - DNA, 64 bp, Artificial sequence
  SiRNA sense oligonucleotide
gatccccgttcctgagcctggactacttcaagagagtagtccaggctcaggaactttttggaaa

SEQ ID NO: 16 - DNA, 64 bp, Artificial sequence
  SiRNA anti-sense oligonucleotide
agcttttccaaaaagttcctgagcctggactactctcttgaagtagtccaggctcaggaacggg

SEQ ID NO: 17 - DNA, 34 bp, Artificial sequence
  Single strand synthetic DNA oligonucleotide
ataacttcgtatagcatacattatacgaagttat

SEQ ID NO: 18 - DNA, 34 bp, Artificial sequence
  Single strand synthetic DNA oligonucleotide
ataacttcgtataatgtatgctatacgaagttat

SEQ ID NO: 19 - DNA, 1975 bp, Mus musculus
ctaaacattcggaggcatttctgtcccctatgccctagttctctcagttgtctttcattctgacttcggtgcttaaaagtccagcttctcggaatcggtagcaaacctctgtgagccggcgtggaacaggaagtgagtacagttctggggagcgccggcccgggctggaggctcggaaagcccaagccagcggaggcctcgcccgagctggagttgtgaccggcgcggcaggtactcggccacaggttacagctcttctacctcttgataagaatggatttccagagttgtctttatgctattgctgaagaactgggcagtgaagacctggctgccctcaagttcctgtgcttggactacatcccacacaagaagcaggagaccatcgaggatgcccagaagctatttctgaggctgcgggaaaaggggatgttggaggaaggcaatctgtctttcctgaaagagctgcttttccacatcagtcggtgggacctgctggtcaacttcctagactgcaaccgagaggagatggtgagagagctgcgggatccagacaatgcccagatttctccctacagggtcatgctctttaagctctcagaagaagtgagcgagttggaattgagatcttttaagttccttttgaacaatgagatccccaaatgtaagctggaagatgacttgagcctgcttgaaatttttgtagaaatggagaagaggaccatgctggcagaaaataacttggaaaccctaaaatcaatctgtgaccaggtcaacaagagcctgctggggaagatcgaggattatgaaagatcaagcacagagagaagaatgagccttgaaggaagggaagagttgccaccttcagttttggatgagatgagcctcaaaatggcggaactgtgtgactcgccaagagaacaagacagtgagtcacggacttcagacaaagtttaccaaatgaagaacaaacctcggggatactgtctgatcatcaacaatcatgatttcagcaaggcccgggaagacataacccaactccgaaaaatgaaggacagaaaaggaacagactgtgataaagaggctctgagtaagacctttaaggagcttcattttgagatagtatcttacgacgactgcactgcaaatgaaatccacgagattctagaaggctaccaaagcgcagaccacaagaacaaagactgcttcatctgctgtatcctatcccacggtgacaagggtgtcgtctatggaacggatgggaaggaggcctccatctatgacctgacatcttacttcactggttcaaagtgcccttccctgtctgggaaacccaagatctttttcattcaggcttgccaaggaagtaacttccagaaaggagtgcctgatgaggcaggcttcgagcaacagaaccacactttagaagtggattcatcatctcacaagaactatattccggatgaggcagactttctgctgggaatggctacggtgaagaactgcgtttcctaccgagatcctgtgaatggaacctggtatattcagtcactttgccagagcctgagggaaagatgtcctcaaggagatgacattcttagcatcctgactggcgtgaactatgacgtgagcaataaagacgacaggaggaacaagggaaagcagatgccacagcccaccttcacactacggaagaagctcttcttccctccctaatgatgtgtgctctccacagttcacatggcttatctgtgcacttttgtgtggatgagtctaatttattttttagaatttcttttgcttttgaatttacatttacataattttcccttttcttccctttaaacccttctttgttatgttccaatttcaaatacatggcctcttttctcattaactgttgtacacacacatacatacacacacacacacacacacacacatttctaaatataacctgtatactatcacttgt

SEQ ID NO: 20 - protein, 420 aa, Homo sapiens
SFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 21 - DNA, 2827 bp, Homo sapiens
gattctgcctttctgctggagggaagtgttttcacaggttctcctccttttatcttttgtgttttttttcaagccctgctgaatttgctagtcaactcaacaggaagtgaggccatggagggaggcagaagagccagggtggttattgaaagtagaagaaacttcttcctgggagcctttcccacccccttccctgctgagcacgtggagttaggcaggttaggggactcggagactgcgatggtgccaggaaagggtggagcggattatattctcctgccttttaaaaagatggacttcagcagaaatctttatgatattggggaacaactggacagtgaagatctggcctccctgaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaagagagaagcagcagccttgaaggaagtcctgatgaattttcaaatggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgatgtatcctctcccatggagacaaaggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcacgccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttacgcttaaggctttgggaatgtttttagctggtggcaataaataccaattcagattgttatctatcactttcaaagctaaattccacggctcatgtctataatcccaaagaggtccaaggctgcagtagcaagaccccgtccccaaatttaaaaattcaaatgatttccttcttattgcttttatgaaatgctctggggtctttttataaatcattaggaattaagtgacacgtacaaaatccagctatgaatatagagggcttatgactataagcccactgttaatattctattaactttaattctactaccacattaaaaaaattagaaagtagccacgtatggtgcactttgggaggttgaggtgggaggatttgcttgaacccgagccatgttcacaccgctgcactcaagcttgggtgacagaaaattttttttttaataaacccaaatttgtttgaaaactttacaagttttaaataagctctccccaaacttgctttatgtatatatatgcttggctaactatatttgctttttgctaactgcatttgcatttgctctttcatctctgcttggattattttatctttaaaatttaagtatcttttttccaaaacatttttaaaaaaa

SEQ ID NO: 22 - protein, 528 aa, Homo sapiens
  misc_feature (339)..(339): Xaa can be any naturally occurring amino acid
MDFSRNLYDIGEGINLDSEDLASLKFLSLDYIPQRKQEPIKDALMLFGINRLGINEKRMLEESNLSFLKELLFRINRLDLLITYLNTRKEEMERELGINTPGIYRAQISAYRVMLYGINISEEVSRSELRSFKFLLQEEISKCKLDDDMNLLDIFIEMEKRVILGEGIYKLDILKRVCAGININKSLLKIINDYEEFSKGIYEELCGVNETTISDSPREGINDSESQTLDKVYGINMKSKPRGYCLIINNHNFAKAREKVPKLHSIRDRNGTHLDAGALTTTFEELHFEIKPHDDCTVEQIYEILKIYGINLMDHSNMDCFICCILSHGIYDKGIITYXGIYTDGIYGINEAPIYELTSQFTGIYLKCPSLAGKPKVFFIGINACGINGDNYGINKGIPVAIETDSEEGINPYLEMDLSSPQTRYIPDEADFLLGIYMATVAINNCVSYRNPAEGTWYIGINSLCGINSLRERCPRGIYDDILTILTEVAINYEVSNKDDKKNMGIYKGINMPQPTFTLRKKLVFPSD

SEQ ID NO: 23 - DNA, 2559 bp, Homo sapiens
ccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaaaggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccactcactttgcaagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcacgccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggatttgcttgaacccaagaggtccaaggctgcagtgagccatgttcacaccgctgcactcaagcttgggtgacagagcaagaccccgtccccaaaaaaattttttttttaataaacccaaatttgtttgaaaacttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttccaaaacattttttaatagaataaaatataatttgatcttaaaaaaaaaaaaaaaaaaaaaaaaaaaaaaaaaaaa

(19) United States
(12) Patent Application Publication
(10) Pub. No.: US 2009/0297515 A1
(43) Pub. Date: Dec. 3, 2009

(54) Use of Caspase-8 Inhibitors for Modulating Hematopoiesis

(75) Inventors: David Wallach, Rehovot (IL); Eugene Varfolomeev, Rehovot (IL); Yael Pewzner-Jung, Rehovot (IL); Tae-Bong Kang, Rehovot (IL); Tehila B. Moshe, Rishon Le Zion (IL)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: YEDA RESEARCH AND DEVELOPMENT CO., LTD., Rehovot (IL)

(21) Appl. No.: 12/472,039

(22) Filed: May 26, 2009

Related U.S. Application Data
(63) Continuation of application No. 10/575,915, filed on Mar. 12, 2007, now abandoned.

(30) Foreign Application Priority Data
Oct. 26, 2003 (IL) 158599

(51) Int. Cl. A61K 39/395; A61K 38/43; A61K 48/00; A61K 38/48

(52) U.S. Cl. 424/133.1; 424/130.1; 424/94.1; 514/44 A; 424/94.63; 424/141.1

(57) ABSTRACT

A method of inhibiting hematopoiesis in a subject is provided. The method is effected by downregulating an expression or activity of caspase-8 in the subject thereby inhibiting hematopoiesis therein.

### FIELD OF THE INVENTION

The present invention relates to methods and articles of manufacture for modulating hematopoiesis and, more particularly, to methods and articles of manufacture which can be utilized for treating disorders characterized by hyper-proliferation of hematopoeitic cells, such as for example, leukemia.

### BACKGROUND OF THE INVENTION

The morphologically recognizable and functionally capable cells circulating in blood include erythrocytes, neutrophilic, eosinophilic, and basophilic granulocytes, B-, T-, non B-, non T-lymphocytes, and platelets. These mature hematopoietic cells derive from and are replaced, on demand, by morphologically recognizable dividing precursor cells for the respective lineages such as erythroblasts for the erythrocytes series, myeloblasts, promyelocytes and myelocytes for the granulocyte series, and megakaryocytes for the platelets. The precursor cells arise from more primitive cells that can be simplistically divided into two major subgroups: stem cells and progenitor cells (for review, see Broxmeyer, H. E., 1983, CRC Critical Review in Oncology/Hematology 1:227-257).

Uncontrollable proliferation or hyperplasia of hematopoietic cells is associated with a variety of life threatening diseases, most notable of which being leukemia. Leukemia is a malignant cancer of the bone marrow and blood. It is characterized by the uncontrolled proliferation and growth of blood cells. The common types of leukemia are divided into four categories: acute or chronic myelogenous, involving the myeloid elements of the bone marrow (white cells, red cells, megakaryocytes) and acute or chronic lymphocytic, involving the cells of the lymphoid lineage.

Acute leukemia is a rapidly progressing disease that results in the massive accumulation of immature, functionless cells (blasts) in the marrow and blood. As a result of this proliferation, the marrow can no longer produce enough normal red and white blood cells and platelets. As a result, individuals suffering from acute leukemia are anemic, sensitive to infections and exhibit defective coagulation processes which can result in uncontrollable bleeding. In contrast, chronic leukemia progresses more slowly and leads to unregulated proliferation and hence marked overexpansion of a spectrum of mature (differentiated) cells.

Standard treatment for leukemia usually involves chemotherapy, radiotherapy and/or bone marrow transplantation. The two major types of bone marrow transplants are autologus (uses the patient's own marrow) and allogeneic (uses marrow from a compatible donor). Radiation therapy, which involves the use of high-energy rays, is usually administered prior to bone marrow transplantation in order to kill all leukemic cells. Chemotherapy in leukemia usually involves a combination of two or more anti-cancer drugs. New treatments for leukemia also include the reversal of multidrug resistance, involving the use of agents which decrease the mechanisms allowing the malignant cells to escape the damaging effects of the chemotherapeutic agent (and leads to refractoriness or relapses); and biological therapy, using monoclonal antibodies, in which toxins are attached to antibodies that react with the complementary antigen carried by the malignant cells; or cytokines such as. interferons, interleukins. Recently, the drug Gleevec (Novartis) has been approved by the FDA for treating chronic myeloid leukemia (CML).

Overall, treatment of leukemia is very complex and depends upon the type of leukemia and condition of the patient. Patients who are resistant to therapy exhibit low survival rates, regardless of when resistance occurs.

In addition, leukemia patients often suffer from critical hyperproliferation of hematopoietic cells, which considerably lowers the efficacy of presently available therapies. It is thus anticipated that repressing hematopoiesis in leukemia patients prior to, or combined with other therapies, can effectively improve the outcome of treatments.

However, despite improvements in outcome with current treatment programs, the search for novel approaches for the treatment of all types of leukemia continues.

There is thus a widely recognized need for, and it would be highly advantageous to have, novel therapeutic approaches for treating leukemia and various other disorders associated with abnormal hematopoeitic processes.

### SUMMARY OF THE INVENTION

According to one aspect of the present invention there is provided a method of inhibiting hematopoiesis in a subject, including downregulating an expression or activity of caspase-8 in the subject thereby inhibiting hematopoiesis therein.

According to another aspect of the present invention there is provided a method of inhibiting hematopoiesis in a subject, including downregulating an expression or activity of at least one polypeptide participating in the caspase-8 signaling pathway in the subject, thereby inhibiting hematopoiesis therein.

According to yet another aspect of the present invention there is provided a method of treating a disorder characterized by hyperproliferation of hematopoeitic cells, including downregulating an expression or activity of caspase-8 in the hematopoeitic cells of a subject having the disorder, thereby treating the disorder characterized by hyperproliferation of the hematopoeitic cells.

According to still another aspect of the present invention there is provided a method of generating an hematopoietic cell population suitable for bone marrow replacement therapy. The method is effected by isolating hematopoietic cells from a subject, and exposing the hematopoietic cells to a molecule capable of downregulating an expression or activity of caspase-8 in the hematopoietic cells, thereby generating an hematopoietic cell population suitable for the bone marrow replacement therapy.

According to an additional aspect of the present invention there is provided a method of treating a disorder characterized by hyperproliferation of hematopoeitic cells. The method is effected by (i) isolating the hematopoietic cells from a donor, (ii) exposing the hematopoietic cells to a molecule capable of downregulating an expression or activity of caspase-8 in the hematopoietic cells, and (iii) transplanting the hematopoietic cells into a recipient, thereby treating a disorder characterized by hyperproliferation of hematopoeitic cells.

According to yet an additional aspect of the present invention there is provided an article-of-manufacture which includes packaging material and a pharmaceutical composition identified for use in modulating hematopoiesis being contained within the packaging material. The pharmaceutical composition includes, as an active ingredient, an agent capable of modifying an activity or an expression of caspase-8 in a subject and a pharmaceutically acceptable carrier.

According to further features in preferred embodiments of the invention described below, downregulating the expression or activity of caspase-8 is effected by an agent selected from the group consisting of (i) a molecule which binds caspase-8, (ii) an enzyme which cleaves caspase-8, (iii) an antisense polynucleotide capable of specifically hybridizing with an mRNA transcript encoding caspase-8, (iv) a ribozyme which specifically cleaves transcripts encoding caspase-8, (v) a small interfering RNA (siRNA) molecule which specifically cleaves caspase-8 transcripts; (vi) a non-functional analogue of at least a catalytic or binding portion of caspase-8, and (vii) a molecule which prevents caspase-8 activation or substrate binding.

According to still further features in the described preferred embodiments the molecule which binds caspase-8 is an antibody or antibody fragment.

According to still further features in the described preferred embodiments the antibody fragment is a Fab or a ScFv fragment.

According to still further features in the described preferred embodiments the molecule which binds caspase-8 is a caspase-8 inhibitor selected from the group consisting of z-VAD-fmk, IEDT-fmk and DEVD-fmk.

According to still further features in the described preferred embodiments the sequence of the small interfering RNA (siRNA) molecule is set forth by SEQ ID NO:15.

According to still further features in the described preferred embodiments the at least one polypeptide is selected from the group consisting of CASP3, CASP4, CASP6, CASP7, CASP9 and CASP10.

According to still further features in the described preferred embodiments the wherein a sequence of said antisense polynucleotide is set forth by SEQ ID NO: 16.

According to still further features in the described preferred embodiments the disorder is selected from the group consisting of acute myelogenous leukemia, acute molymphocytic leukemia, acute lymphocytic leukemia, acute prolymphocytic leukemia, acute lymphoblastic leukemia, chronic lymphocytic leukemia, chronic myeloid leukemia and moldering leukemia.

According to still further features in the described preferred embodiments, treating a disorder characterized by hyperproliferation of hematopoeitic cells further includes chemotherapy.

According to still further features in the described preferred embodiments, treating a disorder characterized by hyperproliferation of hematopoeitic cells further includes radiotherapy

According to still further features in the described preferred embodiments, treating a disorder characterized by hyperproliferation of hematopoeitic cells further includes exposing the hematopoietic cells to one or more growth stimulating factors.

According to still further features in the described preferred embodiments treating a disorder characterized by hyperproliferation of hematopoeitic cells further includes bone marrow transplantation.

According to still further features in the described preferred embodiments the bone marrow transplantation is autologous.

According to still further features in the described preferred embodiments the donor is the recipient.

The present invention successfully addresses the shortcomings of the presently known configurations by providing methods and articles of manufacture for controllably modulating hematopoiesis and thus enabling effective treatment of disorders characterized by hyper-proliferation of hematopoeitic cells.

In addition the present invention provides the use of a downregulator of caspase-8 according to the invention, in the manufacture of a medicament for the treatment of a disorder characterized by hyperproliferation of hematopoeitic cells and or for inhibiting hematopoiesis.

The present invention teaches also, the use of a downregulator of at least one polypeptide participating in the caspase-8 signaling e.g. CASP3, CASP4, CASP6, CASP7, CASP9 and CASP10, in the manufacture of a medicament for the treatment of a disorder characterized by hyperproliferation of hematopoeitic cells and/or for inhibiting hematopoiesis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c illustrate the generation of a conditional caspase-8 knockout mouse. FIG. 1a is a schematic representation of the Casp8 targeting construct, marking the caspase-8 gene exons by black boxes and the positions of restriction sites by vertical lines. FIG. 1b presents the Southern blot analysis of tail DNA obtained from offsprings of Casp8^{fl/+} mice crossed with Casp8^{+/-} mice; the DNA was digested with EcoRV, fractionated on 0.8% agarose gel, blotted onto a nylon membrane, and hybridized with a 5′ probe of 0.9 kb located upstream of exon 1. FIG. 1c presents the Southern blot and PCR analysis of tail DNA obtained from offsprings of Casp8^{fl/+} mice crossed with general Cre-Casp8^{+/-} transgene mice. For Southern blot analysis, the DNA was digested with EcoRV, fractionated on 0.8% agarose gel, blotted onto a nylon membrane, and hybridized with a 3′ probe 0.6 kb located between exon 5 and 6 For PCR analysis, the DNA was amplified using the primers set forth in SEQ ID NOs:1-2. The Southern blot and PCR analyses indicate that only floxed mice which expressed Cre (as shown by PCR to tail DNA) showed a caspase-8 deletion (indicated by a 6.8 Kb fragment), thus indicating a Cre mediated deletion of the loxP flanked Casp8 locus.

FIGS. 2a-d illustrate the effect of caspase-8 deletion on the development of hematopoietic precursor cells in vitro. FIG. 2a presents PCR (picture) and real-time PCR analyses of bone marrow (BM) cells. The BM cells were obtained from Mx1-Cre/Casp8^{fl/+} and Mx1-Cre/Casp8^{fl/-} mice, which had been either treated with pl-pC to induce Cre expression (w. pl-pC), or untreated (w/o pl-pC). FIG. 2b is a bar graph illustrating the total number of colonies developed in vitro from BM cells obtained from Mx1-Cre/Casp8f^{l/+} mice (solid bars) and from Mx1-Cre/Casp8^{fl/-} mice (open bars), which had been either treated or untreated with IFN-α to induce Cre expression. FIG. 2c is a photograph illustrating myeloid colonies which developed in vitro from BM cells obtained from Mx1-Cre/Casp8^{fl/+} and Mx1-Cre/Casp8^{fl/-} mice, which had been treated with pI-pC. FIG. 2d is a bar graph illustrating the number of myeloid colonies and pre-B cell colonies developed in vitro from BM cells obtained from Mx1-Cre/Casp8f^{l/+} mice (solid bars) and from Mx1-Cre/Casp8^{fl/-} mice (open bars), which had been treated with pI-pC (Total, Total number of myeloid colony-forming units); BFU-E, erythroid forming units; CFU-GM, granulo-macrophagic colony-forming units; CFU-Mix, granulocytic-erythroid-megacaryocytic-macrophagic colony-forming units).

FIGS. 3a-c illustrate the effect of caspase-8 knock-out on the capacity of BM (bone marrow) cells to develop in spleen. FIG. 3a is a photograph illustrating the general appearance of spleens of the recipient mice reconstituted with BM cells of pIpC-injected Mx1-Cre/Casp8^{fl/+} (fl/+) and Mx1-Cre/Casp8^{fl/-} (fl/−), and non-reconstituted control mice (con). FIG. 3b is a bar graph illustrating the total number of colonies in spleens of the recipient mice with BM cells of pIpC-injected Mx1-Cre/Casp8^{fl/+} mice (open bars) and of Mx1-Cre/Casp8^{fl/-} mice (solid bars). FIG. 3c is a bar graph illustrating the weight of spleens of the recipient mice with BM cells of pIpC-injected Mx1-Cre/Casp8^{fl/+} mice (light gray bars) and Mx1-Cre/Casp8^{fl/-} (dark gray bars) and of non-reconstituted control mice (black bars).

FIGS. 3d-e illustrate the cell-autonomous role of caspase-8 on the hematopoietic precursor function. FIG. 3d is a bar graph illustrating the total number of myeloid colonies in the culture of BM cells of irradiated Mx1-Cre/Casp8^{fl/+} (solid bars) and Mx1-Cre/Casp8^{fl/-} mice (open bars), and which have been injected with pI-pC (3 times, once or none), transplanted with BM cells obtained from normal C57BL/6 (Ly-5.1) mice. FIG. 3e is a bar graph illustrating the total number of myeloid colonies in the culture of BM cells of irradiated C57BL/6 (Ly-5.1) mice and transplanted with BM cells obtained from Mx1-Cre/Casp8^{fl/+} (solid bars) and from Mx1-Cre/Casp8^{fl/-} mice (open bars), and which have been injected with pI-pC (3 times, once or none).

FIGS. 4a-e illustrate phynotypic and genotypic analyses of bone marrow (BM) cells of CD19 Cre/Casp8 knock-out (F/−) and control (F/+) mice. FIGS. 4a-d are FACS analyses of propidiumiodide (PI) negative (live cells) BM B cells using antibodies to B220, IgM and CD43 to define Pro-B (B220^{low}/IgM⁻/CD43⁺), Pre-B (B220^{low}/IgM⁻/CD43⁻), Immature (B220^{low}/IgM⁺/CD43⁻) and mature re-circulating B (B220^{high}/IgM⁺/CD43⁻) cells. The different BM B cell populations were gated by CellQuest analysis software, and percentage of each population was defined. The results (FIGS. 4a-b) indicate a reduction in the percentage of re-circulating mature B cells in CD19 Cre/Casp8 knock-out (F/−) as compared with control (F/+) mice. The different populations of BM B cells were sorted by FACSVantage and PCR analysis was done directly on the sorted cells to define the level of the deleted caspase-8 allele (FIG. 4e). This PCR analysis demonstrate that the deletion occurs mainly between Pre-B and Immature B cell stages reaching a maximum deletion of about 90-95% in the mature B cell stage. No difference was found between the deletion rate in CD19 Cre/Casp8 knock-out (F/−) as compared with control (F/+) mice.

FIGS. 5a-e illustrate phenotypic and genotypic analyses of spleen cells of CD19 Cre/Casp8 knock-out (F/−) and control (F/+) mice. FIGS. 5a-d are FACS analyses of live splenocytes stained with antibody to CD3, B220 and IgM. These results suggest that in CD19 Cre/Casp8 knock-out (F/−) spleen B cells there are more cells which are B220+/IgM^{low-neg} (FIGS. 5c-d). This may indicate that Caspase-8 is essential factor in specific B cell subsets. FIG. 5e is a PCR analysis of DNA extracted from purified splenic B cells showing that in CD19 Cre/Casp8 knock-out (F/−) as well as in control (F/+) mice there is high level of deletion of the caspase-8 allele on the DNA level.

FIGS. 6a-g illustrate phenotypic and genotypic analyses of stimulated purified splenic B cells of Casp8 knock-out (F/−) and control (F/+) mice. FIGS. 6b-g illustrate FACS analyses of CFSE labeled splenic B cells after 4 days of IgM (FIGS. 6b-c), CD40 (FIGS. 6d-e), and LPS (FIG. 6f-g) stimulation. These results demonstrate that upon LPS stimulation CD19 Cre/Casp8 knock-out (F/−) B cells are defective in their proliferation capacity. FIG. 6a demonstrate PCR analysis of 4 days stimulated purified splenic B cells, and shows that the level of deletion of the caspase-8 allele after stimulation of cells is similar to the level of deletion in naïve primary splenocytes (FIG. 5e).

FIGS. 7a-f illustrate the effect of caspase-8 gene deletion on macrophage precursors differentiation. FIG. 7a shows the extent of cell adherence after 7 day culturing of BM cells of pI-pC injected Mx1-Cre/Casp8^{fl/+} and Mx1-Cre/Casp8^{fl/-} mice in the presence of M-CSF. FIGS. 7b-e show features of macrophages generated by culturing BM cells of LysM-Cre/Casp8^{fl/+} and LysM-Cre/Casp8^{fl/-} mice with M-CSF. FIG. 7b illustrates the appearance of the adherent cells after culturing for 7 days. FIG. 7c shows the yield of adherent cells (by MTT assay) after culturing for the indicated periods; white bars: cells of fl/+ mice; black bars; cells of fl/− mice. FIG. 7d illustrates the extent of deletion of the floxed caspase-8 allele as assessed by PCR (top) and real-time PCR (numbers at the bottom) in cells found to be attached to the culture plate after the 7 day culturing period. FIG. 7e shows a FACS analysis of the non-attached cells after a 5-day culturing period. Analysis illustrates expression of the macrophage marker CD11b and presence of the annexin-V marker. In multiple tests, the extent of dead cells observed in the LysM-Cre/Casp8^{fl/-}-derived cultures was significantly higher than in the LysM-Cre/Casp8^{fl/+}-derived cultures. FIG. 7f shows the extent of the floxed caspase-8 allele deletion in peritoneal macrophages derived from LysM-Cre/Casp8^{fl/+} mice and from LysM-Cre/Casp8^{fl/-} mice.

### DESCRIPTION OF THE PREFERRED EMBODIMENTS

The present invention relates to methods and articles of manufacture which can be used for treating hematopoiesis related disorders. Specifically, the present invention relates to modulating hematopoiesis by regulating the expression or activity of caspase-8 in hematopoietic cells.

The principles and operation of modulating hematopoiesis according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Enzymes of the caspase (cysteine aspartase protease) family play central roles in the initiation of programmed death in eukaryotic cells (1). The caspase enzymes cleave specific protein substrates thus either ablating or triggering the proteins function. Caspases are known to be capable of initiating a set of functional and structural changes that may lead to an apoptotic death. These caspases can be selectively activated by specific inducers, allowing initiation of the death program by a variety of different external and intracellular signals. Recently, several studies suggested that certain caspases might also serve non-apoptotic functions (2-8).

Caspase-8 (see GeneCard GC02P200822 at http://www.rzpd.de/cards/index.html for extensive description) is known for its role in cell death induction via ligands of the TNF family. This caspase binds through two tandem N-terminal death-effector domains to an adapter protein called MORT1/FADD that in turn binds either directly to the intracellular domains of receptors of the TNF/NGF family or to other adapter proteins that bind to the receptors. Activation of caspase-8 through these associations has been shown to initiate the death process that these receptors induce (9-10, 26; Salmena L et al. Genes Dev. 17:883-895, 2003; Olson N. E. et al. J. Immunol. 170:6065-6072, 2003). Recently, Chan et al. (Nature 419:395, 2002) identified a caspase-8 mutation in patients with autoimmune lymphoproliferative syndrome-related symptoms. T, B, and NK cells from these patients exhibited defects in activation suggesting that caspase-8 plays in lymphocyte activation. Olson et al, (The Journal of Immunology 170: 6065-6072, 2003) reported that caspase-8 increased B cells activation by a variety of stimuli, and that inhibitors selective for caspase-8 blocked B cell proliferation. It was further suggested that caspase-8 activity is required for stimulated B lymphocytes to enter the cell cycle. Knockout of the caspase-8 gene is lethal in utero, as reported by Varfolomeev et al. (11), suggesting that this enzyme is critical for embryonal development, but curtailing its use for assessing the role of caspase-8 in adult mice.

While reducing the present invention to practice, the present inventors have unexpectedly discovered that knockout of the caspase-8 gene in hematopoeitic cells of mice led to a surprising and unexpected discovery indicating a central role of caspase-8 in hematopoiesis. As is illustrated in Examples 2-7 of the Examples section which follows, absence of caspase-8 in hematopoeitic cells of adult mice substantially impaired bone-marrow hematopoietic precursor cells production and substantially reduced the capacity of bone marrow cells to colonize spleens of irradiated mice (Example 3). Furthermore, caspase-8 knockout substantially reduced the capacity of bone marrow cells to reconstitute in irradiated chimera mice (Example 4). In addition, caspase-8 knockout compromised the activation of B lymphocytes by stimulants (Example 5), inhibited differentiation of monocyte precursors to macrophages (Example 6), and impaired embryonic hematopoiesis (Example 7). Overall, these results clearly demonstrate that caspase-8 is essential for the formation and development of hematopoeitic cells.

Although several studies have suggested that caspases are involved in non-apoptotic functions (2-8, 26), none of these reports have described or suggested any involvement of caspase-8 in hematopoiesis. Recently, Varfolomeev et al. (11) reported that homozegous knockout of the caspase-8 gene (Casp8) in mice was lethal in utero and that the embryos manifested impaired heart muscle development and congested accumulation of erythrocytes. However, the non-apoptotic effects of caspase-8 in adult mice could not be uncovered using the homozygous caspase-8 knockout mice of Varfolomeev et al.

Thus, according to one aspect of the present invention, there is provided a method of inhibiting hematopoiesis. The method is effected by downregulating an expression or activity of caspase-8 in hematopoeitic cells, thereby inhibiting hematopoiesis therein.

As used herein, the term “hematopoiesis” refers to the formation and development of blood cells involving proliferation and/or differentiation from stem cells. Although hematopoiesis takes place in vivo (i.e. in bone marrow of a subject), as is further described herein below, inhibition of hematopoiesis can be effected by both in-vivo and ex-vivo (in-vitro) approaches as is further described herein below.

As used herein the phrase “inhibiting an expression or activity” refers to partially or fully inhibiting expression (transcription and/or translation) or activity (e.g., enzymatic or ligand binding) of caspase-8. Preferably inhibition of caspase-8 expression according to the present invention targets splice variants alpha 1 and alpha 2 of caspase-8 (SEQ ID NOs: 21 and 23 respectively) while inhibition of activity targets the polypeptides encoded by these sequences (SEQ ID NOs: 20 and 22 respectively).

Several different approaches can be used to down regulate activity of caspase-8 in hematopoietic cells.

For example, inhibiting caspase-8 activity can be achieved by an agent such as an antibody or an antibody fragment capable of specifically binding caspase-8 and modified in a way that will allow it to enter the cell. Preferably, the antibody specifically binds at least one epitope of caspase-8. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Preferred epitopes of caspase-8 are those comprising the catalytic site (e.g around cystein 360) or the region of association of caspase-8 with the adapter protein FADD (that is, the death effect or domain region that extends from amino acid 2 till 183)

The term “antibody” as used herein includes intact molecules as well as functional fragments thereof, such as Fab, F(ab′)2, and Fv. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). Commercially available polyclonal and monoclonal antibodies that bind to caspase-8, and suitable for use in the present invention are disclosed in Linscott's Directory of Immunological and Biological Reagents, 12th Edition, 2002-2003, Publisher: W.D. Linscott, Petaluma, Calif. These include monoclonal antibody clone Nos. 1186 (IgG1), IC12 (IgG1), 8CSPO1 (IgG1), 8CSPO2 (IgG1), 8CSPO3 (IgG1), FLICE1 (IgG1), FLICE3 (IgG1), FLICE4-1-20 (IgG1), 89-2 (IgG2a), CAS8 (IgM), 11G10 (cleaved caspase-8), 843.11 (human caspase-8), IH10E4H10 (human caspase-8 IgG2a-RAT), 12F5 (human caspase-8, IgG2b), 5D3 (human caspase-8, IgG2b), 5F7 (human caspase-8, IgG2b), S5FLG (human caspase-8, IgG2b), 57F (human caspase-8, IgG2b), 79A1337 (human caspase-8 176-460 fragment, IgG), and 5D3 (human/mouse caspase-8 180-460 fragment, IgG2b).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (scFv) are prepared by constructing a structural gene comprising DNA sequences encoding the VH and VL domains connected by an oligonucleotide. The structural gene is inserted into an expression vector, which is subsequently introduced into a host cell such as E. coli.

Since caspase is a cytosolic enzyme, the antibody utilized by the present invention is preferably an antibody fragment which is capable of being delivered to, or expressed in, hematopoetic cells. Thus, an scFv Ab coding sequence is preferably included in an vector suitable for expression of the anti-caspase-8 scFv fragment in hematopoietic cells (see hereinbelow for details on expression vector construction). A suitable scFv expression vector can be, for example, pIG6 [Ge: in Antibody Engineering (Boreback C. A. K ed.) 2nd ed. pp 229-261, 1995 Oxford university], pFab5c or pcDNA3.1 is described by Khoshar (PNAS 99:1002-1007, 2002).

The recombinant host cells synthesize a single polypeptide chain with a linker peptide bridging the two V domains. Methods for producing scFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Inhibition of caspase-8 activity can also be effected by utilizing known peptide inhibitors of caspase-8 such as, for example z-VAD-fmk, IEDT-fmk, DEVD-fmk, or by any peptide inhibitor derived from a polypeptide sequence capable of interacting with the catalytic site of caspase-8 (e.g., substrate analogue), or molecules derived from the viral caspase inhibiting protein Crm-A and p35, or molecules derived from the cellular inhibitor of caspase-8 cFLIP or from its various viral homologues. Description of suitable biochemical/molecular approaches which can be utilized for identifying additional inhibitors is provided hereinbelow.

Additional inhibitors of caspase-8 can be identified using molecular design to approach, utilizing on the three-dimensional molecular structure of caspase-8 described by Blanchard et al. (Structure 7:1125-1133, 1999) and by Watt et al. (Structure 7:1135-1143, 1999) and on its substrate binding model which has been created by Chou et al., (FEBS 419:49-54, 1997).

Caspase-8 activity can also be inhibited by a protein relocating caspase-8 to a subcellular organelle/location and rendering it incapable of exerting its biological effect, for example ‘bifunctional apoptosis regulator’ (BAR), a protein dictating localization of caspase-8 in association with the mitochondria (Stegh A H et al. J Biol. Chem. 2002 277:4351-60).

Downregulation of expression of caspase-8 in hematopoietic cells can be effected using any one of several molecular approaches.

For example, caspase-8 transcription can be inhibited via RNA interference by utilizing a small interfering RNA (siRNA) molecule. RNA interference is a two step process; the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase (Hutvagner and Zamore, Curr. Opin. Genetics and Development 12:225-232, 2002).

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002). Synthesis of RNAi molecules suitable for use with the present invention can be effected as follows. First, the Caspase-8 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene. An siRNA sequence which can be used to downregulate caspase-8 expression according to the teaching of the present invention include SEQ ID NO: 15.

Caspase-8 siRNA sequences, suitable for use according to the teaching of the present invention, have been demonstrated capable of preventing acute liver failure in mice (Zender et al., (PNAS 13: 7797-7802, 2003), Systemic application of the siRNA inhibited expression of caspase-8 in the liver, thereby prevented FAS (CD95)-mediated apoptosis. Furthermore, improvement of survival due to RNA interference was significant even when the caspase-8 siRNA was applied during ongoing acute liver failure.

Another agent capable of downregulating a caspase-8 is a DNAzyme molecule capable of specifically cleaving an mRNA transcript or DNA sequence of the caspase-8. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 2002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Inhibition of caspase-8 expression can also be effected by using an antisense polynucleotide capable of specifically hybridizing with an mRNA transcript encoding the caspase-8 thereby specifically inhibiting translation of the caspase-8 transcripts.

Design of antisense molecules which can be used to efficiently inhibit caspase-8 expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft, J Mol Med 76: 75-6, 1998; Kronenwett et al., Blood 91: 852-62, 1998; Rajur et al., Bioconjug Chem 8: 935-40, 1997; Lavigne et al., Biochem Biophys Res Commun 237: 566-71, 1997; and Aoki et al., Biochem Biophys Res Commun 231: 540-5), 1997].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9, 1999].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16, 1374-1375, 1998).

Several clinical trials have demonstrated safety, feasibility and activity of antisense oligonucleotides. For example, antisense oligonucleotides suitable for the treatment of cancer have been successfully used (Holmund et al., Curr Opin Mol Ther 1:372-85, 1999), while treatment of hematological malignancies via antisense oligonucleotides targeting c-myb gene, p53 and Bcl-2 had entered clinical trials and had been shown to be tolerated by patients (Gerwitz Curr Opin Mol Ther 1:297-306, 1999).

More recently, antisense-mediated suppression of human heparanase gene expression has been reported to inhibit pleural dissemination of human cancer cells in a mouse model (Uno et al., Cancer Res 61:7855-60, 2001).

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

The antisense sequences may include a ribozyme sequence which is capable of cleaving transcripts encoding Caspase-8, thereby preventing translational of those transcripts into functional Caspase-8. Such a ribozyme is readily synthesizable using solid phase oligonucleotide synthesis.

Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. 1998 October; 9(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. 1998 Jul. 15; 10(2-3):163-71.]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Although the above describe expressible inhibitors (e.g., antibody fragments, antisense, etc.) can be synthesized using recombinant techniques and provided directly to hematopoietic cells via, for example, injection, such molecules can also be expressed directly in the hematopoeitic cells by utilizing an expression vector which includes a polynucleotide sequence encoding the inhibitor positioned under the transcriptional control of a promoter sequence suitable for directing constitutive tissue specific or inducible transcription in mammalian cells.

Constitutive promoters suitable for use with the present invention include sequences which are functional (i.e., capable of directing transcription) under most environmental conditions and most types of cells such as the cytomegalovirus (CMV) and Rous sarcoma virus (RSV). Tissue specific promoters suitable for use with the present invention include sequences which are functional in hematopoietic cells, example include, for example, the promoter sequences described by Clark and Gordon (Leukoc Biol. 63:153-68, 1998); Stein et al. (Cancer 15:2899-902, 2000); and Hormas et al., (Curr Top. Microbiol. Immunol. 211:159-64, 1996). Inducible promoters suitable for use with the present invention include for example the tetracycline-inducible promoter (Srour et al., hromb. Haemost. 90: 398-405, 2003).

The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the translation efficiency of a polypeptide inhibitor such as Scfv. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can also be used by the present invention. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I).

Recombinant viral vectors are useful for in vivo expression of caspase-8 inhibitors since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Further description of constructs which are suitable for hematopoeitic cell-specific expression is provided in Malik et al. (Blood 15: 86:2993-3005, 1995).

The use of a vector for inducing and/or enhancing the endogenous production of an endogenous inhibitor of caspase-8, in a cell normally silent for expression of an inhibitor, or expressing amounts of inhibitor which are not sufficient, are also contemplated according to the invention. The vector may comprise regulatory sequences functional in the cells desired to express the inhibitor. Such regulatory sequences comprise promoters or enhancers. The regulatory sequence is then introduced into the right locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

It will be understood by the person skilled in the art that it is also possible to shut down caspase-8 expression using the same technique, i.e. by introducing a negative regulation element, like e.g. a silencing element, into the gene locus of caspase-8, which will result in down-regulation or prevention of caspase-8 expression. The person skilled in the art will understand that such down-regulation or silencing of caspase-8 expression has the same effect as the use of a caspase-8 inhibitor in order to prevent and/or treat disease.

Various methods can be used to introduce the expression vector of the present invention into hematopoietic cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

It will be appreciated that the expression constructs utilized for expressing the inhibitor are preferably constructed and introduced into hematopoietic cells in a manner which enables exclusive and controllable expression in these cells. For example, by utilizing a viral expression vector which can exclusively transform hematopoetic cells or by transforming such cells ex-vivo, and by utilizing an inducible promoter sequence in the expression construct (see examples above), exclusive and controllable expression in these cells can be achieved.

Such an expression strategy is advantageous in particularly when used in context of leukemia treatment, since it allows for precise control over hematopoiesis and thus regulation of hematopoeitic cell numbers.

Although hematopoiesis is preferably inhibited according to the present invention by utilizing the above described caspase-8 specific inhibitors, it will be appreciated that since caspase-8 is triggered by, and in turn triggers, cellular polypeptides, hematopoiesis can also be inhibited by downregulating expression or activity of such polypeptides. as for example the adapter protein FADD to which has to bind in order to become activated. It may also be inhibited by enzymatically inactive derivatives of caspase-8, such a caspase-8 mutant in which the active-site cystein was replaced with serine.

As is mentioned hereinabove, downregulation of caspase-8 expression or activity may be effected in vitro by exposing cultured hematopoietic cells to a downregulating agent, or in vivo by administering such an agent to a subject. These in vivo and in vitro approaches can be utilized to treat a variety of hematopoiesis related disorders.

Thus, according to another aspect of the present invention, there is provided a method of treating a disorder characterized by hyperproliferation of hematopoeitic cells. The method is effected by downregulating an expression or activity of caspase-8 in the hematopoeitic cells of a subject using any one of the approaches described hereinabove.

As used herein the phrase “disorder characterized by hyperproliferation of hematopoeitic cells” refers to, any disease characterized by abnormal high proliferation rates of one or more types of hematopoeitic cells. Examples include leukemia, such as, acute myelogenous leukemia, acute molymphocytic leukemia, acute lymphocytic leukemia, acute prolymphocytic leukemia, acute lymphoblastic leukemia, chronic lymphocytic leukemia, chronic myeloid leukemia, moldering leukemia, autoimmune diseases, lymphoprofelative disorders and alergies.

When downregulation is effected ex-vivo, hematopoietic cells, are isolated from bone marrow cells, or from the circulation after mobilization of bone marrow cells, of an individual suffering from the hematopoietic disorder and treated with a caspase-8 inhibitor. Isolation of bone marrow cells can be effected by aspiring a small amount of bone marrow from the iliac crest of the individual using a procedure such as described in U.S. Pat. Nos. 4,481,946 and 4,486,188. The isolated bone marrow sample can be cultured or preserved for future use. Preservation of bone marrow cells is preferably effected by freezing in liquid nitrogen, using a procedure such as described in U.S. Pat. Nos. 4,107,937 and 4,117,881. Subsequently, the bone marrow cells are cultured in the presence of a caspase-8 downregulating agent, such as those described hereinabove (e.g., RNAi). Preferably, the cells are grown in standard Nunc culture plates supplemented with Fischers' medium (GIBCO) enriched with fetal bovine serum or human serum and incubated at 37° C. in 5-6% CO₂ atmosphere. While in culture, the cells may also be exposed to growth/differentiating stimulating agents (e.g., IL1-7, IL-9, IL-11, IL13-15, G-CSF, GM-CSF, erythropoietin, thrombopoietin, stem cell factor and flk2/flt3 ligand) in order to stimulate maturation of cells. In any case, once hematopoietic cells exhibit loss of hyperproliferation activity, based on, for example, reduced density of immature blood cells, or caspase activity, they are administered to the recipient, preferably by intravenous infusion. Following transplantation, the recipient is monitored for the reconstitution of the bone marrow cells, based on blood cell and platelet counts using well known monitoring procedures.

Although in vivo downregulation of caspase-8 activity in hematopoietic cells is more difficult to effect, use of inhibitory agents which are directly expressed in hematopoietic cells as described hereinabove or alternatively, which are coupled to targeting moieties, or injected directed into bone marrow, can be utilized to efficiently downregulate caspase-8 in such cells. A direct injection of the inhibitor agent to the bone marrow is preferred over a systemic administration, since it provides rapid dissemination of the inhibitory agent to the target hematopoietic cells. Administering of the agent by injection to the bone marrow is preferably performed using a releasable extending and retracting needle such as described in U.S. Pat. No. 5,451,210.

Alternatively, the caspase-8 inhibitory agent can be delivered to the target cells by a liposome carrier. The term “liposome” used herein refers to a spherical particle in an aqueous medium, formed by a lipid bilayer enclosing an aqueous compartment. Liposomes have been used effectively for a variety of therapeutic purposes, in particular, for carrying therapeutic agents to target cells by systemic administration. Preferably, the caspase-8 inhibitor is entrapped in a liposome such as described in U.S. Pat. No. 6,043,094. The preferred liposome includes outer surfaces that contain an affinity moiety selected capable of specifically binding the surface hematopoietic cells, such as described, for example CD34 ligand, GM-CSF receptor ligand, IL-4 receptor ligand, MU-1 hematopoietin receptor ligand, or a CD33 ligand such as described in U.S. Pat. No. 6,599,505. The preferred liposome further includes and a hydrophilic polymer-coating which is capable of shielding the affinity moiety from interaction with the target surface. The hydrophilic polymer coating is made up of polymer chains covalently linked to surface lipid components in the liposome through releasable linkages. After a desired liposome biodistribution is achieved, a releasing agent is administered to cause cleaving of a substantial portion of the releasable linkages in the liposomes, to expose the affinity agent to the target surface.

Alternatively, the caspase-8 inhibitor can be entrapped in a fusogenic liposome, such as described in U.S. Pat. No. 6,224,904, which is capable of delivering the entrapped inhibitor into the cytoplasm of the target cells. Further description of formulations suitable for in vivo downregulation of caspase-8 activity is provided hereinbelow.

Hematopoietic disorder treatment according to the teachings of the present invention is preferably combined with standard chemotherapy and/or radiotherapy to effectively destroy leukemia cells. The treatment may also be followed by providing the subject with one or more growth stimulating factors so as to stimulate the recovery of healthy hematopoietic cells. Suitable growth stimulating agents include cytokines such as IL1-7, IL-9, IL-11, IL13-15, G-CSF, GM-CSF, erythropoietin, thrombopoietin, stem cell factor and flk2/flt3 ligand. Alternatively, or additionally, the treatment can be followed by transplanting bone marrow cells, either autologous or allogeneic, so as to promote new growth of healthy stem cells in the subject, using the procedures described hereinabove.

The above described caspase-8 downregulating agents can be administered directly to the subject, or to isolated hematopoietic cells per se or as a part (active ingredient) of a pharmaceutical composition.

As used herein a “pharmaceutical composition” refers to a preparation of one or more of the active ingredients or agents described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to levels of the active ingredient are sufficient to substantially affect the body weight or fat content of an individual. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Preferably, the pharmaceutical composition is administered in a local rather than systemic manner, for example, via exposing cells, such as hematopoietic cells, ex vivo or by injection of the pharmaceutical composition directly into the bone marrow of a patient. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

Thus, the present invention provides a novel approach for modulating hematopoiesis and for treatment hematopoiesis related diseases.

It will be appreciated that since the present findings implicate caspase-8 as being involved in hematopoeitic cell proliferation, it is conceivable that agents which upregulate expression or activity of caspase-8 can be utilized to promote hematopoiesis in a subject. Accordingly, upregulation of caspase-8 expression levels may be effected by delivering to a subject an exogenous polynucleotide sequence designed and constructed to express at least a functional portion of the caspase-8 in the subject.

In order to generate a polynucleotide construct capable of expressing at least a functional portion of caspase-8, a polynucleotide segment encoding caspase-8 or a portion thereof, can be ligated into an expression vector system suitable for transforming mammalian cells and for directing the expression of caspase-8 within the transformed cells. Description of suitable expression vectors, promoters and methods of transformation is provided hereinabove.

Caspase-8 may further be upregulated by increasing expression of endogenous caspase-8 in the subject or by increasing endogenous caspase activity. This can be done for example by drugs that will impose demethylation of CpG islands in the promoter of caspase-8.

An agent capable of upregulating caspase-8 may be utilized for treating a disorder characterized by hematopoeitic cells deficiency, such AIDS, cancer, cachexia or diabetes. In order to facilitate practice of the methods described hereinabove, and/or production of pharmaceutical compositions and articles of manufacture as described hereinabove, the present invention further provides a method of identifying novel drug candidate for treatment of hematopoiesis related disorders, such as leukemia. The method of identifying a drug candidate includes screening a plurality of molecules for a molecule capable of at least partially inhibiting caspase-8 expression or preferably activity. Screening may be effected using an antibody based inhibition assay, a competitive ligand binding inhibition assay, or an enzymatic activity inhibition assay. Screening is preferably effected by evaluating the catalytic activity of the caspase-8 using a high throughput screening assay such as described in U.S. Pat. No. 6,342,611. Briefly, the assay utilizes a caspase-8 specific substrate labeled with a fluorogenic/fluorescent moiety. When the labeled substrate is exposed to caspase-8, the reporter molecules are cleaved which in turn result in emission of fluorescence. If an inhibitor molecule is also present in the reaction mixture, the level of fluorescence emission is reduced, relative to the enzyme only treatment (negative control) as in a competition assay. The quantitative difference in fluorescence emission can be accurately measured using a standard fluorometer and the method is easily adaptable to perform high throughput screening of candidate caspase-8 inhibitors. For determining specificity of inhibitors, the selected caspase-8 inhibitors are exposed to one or more additional similar assays but which include one or more different caspase enzymes (positive controls) and their respective specific substrate.

The screening for caspase-8 inhibitors can be conveniently effected by using the commercial caspase-8 fluorescent kit, ApoAlert™ (CLONTECH), which detects the cleavage of synthetic caspase-specific substrate, quickly and quantitatively.

Once inhibitors are identified further analysis is conducted in order to determine their cell penetration capabilities and their toxicity to mammals. If need be suitable drug candidates are modified in order to increase cell penetration thereof and decrease toxicity without substantially affecting their caspase-8 inhibitory activity.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984); “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996) and Parfitt et al. (1987). Bone histomorphometry: standardization of nomenclature, symbols, and units. Report of the ASBMR Histomorphometry Nomenclature Committee. J Bone Miner Res 2 (6), 595-610; all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Caspase-8 Conditional Knock-Out Mice

A novel caspase-8 knockout model was developed in order to elucidate the functions of caspase-8 in adult tissues, and to overcome the limitations of previously described caspase-8 knockout models [the knockout of caspase-8 gene is known to be lethal in uteru (11)].

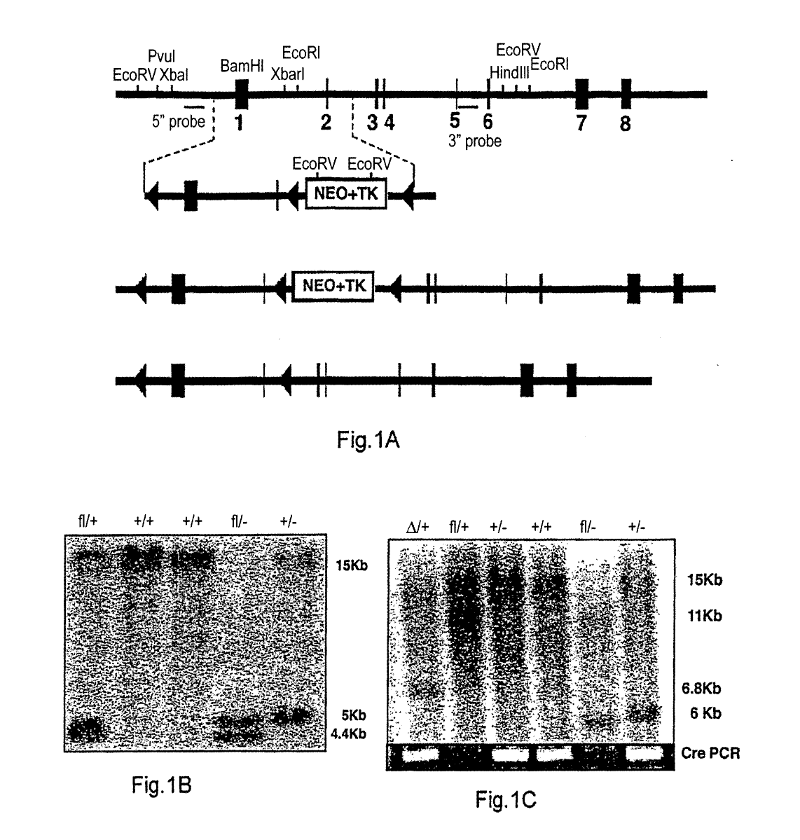

A DNA fragment which includes the mouse caspase-8 gene (Casp8) and adjacent regions was isolated from the mouse caspase-8 of SEQ ID NO: 19 and cloned into a pBluescript vector as previously described by Varfolomeev (11). A Casp8 targeting construct was assembled by inserting a loxP site (SEQ ID NO: 17-18) upstream of the first exon of the caspase-8 gene and a NEOr+TK (thimidine kinase) cassette flanked by two loxP sites downstream of exon 2, as illustrated in FIG. 1a.

The Casp8 targeting construct was linearized with NotI and introduced into R1 embryonic stem (ES) cells by electroporation followed by selection of transformed ES cells with G418. The transformed ES clones were then screened for homologous recombination by Southern blot analysis using genomic DNA probes from regions upstream of the 5′ arm and downstream of the 3′ arm of the targeting construct 5′ probe of 0.9 kb located upstream of exon 1, 3′ probe 0.6 kb located between exon 5 and 6. Positive ES cell clones were then transfected with supercoiled Cre (Cyclization recombination enzyme)-expressing construct (EF1a-GFPcre/pBS500; Gagneten, S., et al., Nucleic Acids Res, 1997. 25: 3326-31, 1997) followed by selection with gancyclovir. Following transfection, the Casp8^{fl/+} ES cells were selected and aggregated with MF-1 blastocysts to generate chimeric mice. The chimeric mice were mated with MF-1 mice to obtain heterozygous offsprings carrying the conditional caspase-8 allele (Casp8^{fl/+}).

Homozygous Casp8^{flox/flox} mice were generated by intercrossing heterozygous casp8^{fl/+} mice. Cre positive caspase-8 negative mice (Cre-Casp8^{+/-}) were generated by crossing Casp8 heterozygous full knockout mice (Casp8^{+/-}) with transgenic Mx1-Cre mice, Tie1-Cre or CD19 Cre [expressing the Cre under the control of alpha/beta interferon (Kuhn et al., Science 269: 1427-1429, 1995), the Tie1 promoter (Gustafsson et al., J. Cell Science 114:671-76, 2001) and the CD19 promoter (Rickert et al. Nuc. Aci. Res. 25:1317-1318, 1997), respectively]. Caspase-8 conditional knock-out mice (Mx1-Cre/Casp8^{fl/-}) were generated by crossing the Casp8^{fl/fl} with the Mx1-Cre/Casp8^{+/-} mice. The Mx1-Cre/Casp8^{fl/+} animals which also resulted from this crossing were used as experimental control litter mates.

To induce the expression of Cre recombinase in Mx1-Cre, mice were injected intraperitoneally with 250 μl of 1 mg/ml double-stranded poly(I)-poly(C) RNA (pI-pC; Sigma, St. Louis, Mo.) once or three times over a two day period.

Genetic screening for the Cre transgene and conditional allele were performed by polymerase chain reaction (PCR) analysis using tail DNA. The oligonucleotide primers utilized in the PCR analysis are shown in Table 1. The Cre genotype was determined by using CRE-specific oligonucleotide primers as shown in Table 1 (SEQ ID NOs:1-2); while Casp8^{fl/+} and Casp8^{fl/-} genotypes were determined by using Casp8-specific oligonucleotide primers as shown in Table 1 (SEQ ID NOs:3-6).

TABLE 1
Oligonucleotide primers and probes
used for PCR analysis

| | | SEQ |
|---|---|---|
| Primer | | ID |
| Sequences | Sequence | NO: |
| Sense for Cre | AGCTGGCTGGTGGCAGATGG | 1 |
| Antisense Cre | CGTTGATGCCGGTGAACGTG | 2 |
| Sense for mutant | TAGCCTCTTTGGGGTTGTTCTACTG | 3 |
| caspase-8 | | |
| Antisense for | TGGGGCTTCGTTTAGTCTGTACTTC | 4 |
| mutant caspase-8 | | |
| Sense for flox | TAGCCTCTTTGGGGTTGTTCTACTG | 5 |

Conditions for the above described amplification reactions were as follows: 5 minutes at 95° C. followed by 33 cycles, each consisting of 45 seconds at 94° C., 30 seconds at 65° C., and 30 seconds at 72° C., with a final cycle of 10 minutes at 72° C. (for Cre); 5 minutes at 95° C. followed by 30 cycles, each consisting of 45 seconds at 94° C., 60 seconds at 60° C., and 60 seconds at 72° C., with a final cycle of 10 minutes at 72° C. (for caspase8^{+/-} and flox).

Southern blot and PCR analyses confirmed that the loxP-flanked caspase-8 allele was deleted only in mice which carry the Cre gene and induced by pI-pC, as illustrated in FIGS. 1b-c.

### Example 2

### The Knock-Out of Caspase-8 in Mice Impairs Hematopoietic Precursor Cells

Materials and Methods:

Animals: The conditional caspase-8 knock-out mice, Mx1-Cre/Casp8^{fl/-} and their control littermates, Mx1-Cre/Casp8^{fl/+}, were generated as described hereinabove.

In vitro assay: Bone marrow (BM) cells were harvested from mice femora and suspended for single cells. Nucleated cells were then counted and the suspension was diluted to 2×10⁵ cells per 1 ml in Iscove modified Dulbecco medium (IMDM) with 2% FBS. For each assay, 2×10⁴ cells were mixed with 1 ml of methylcellulose media containing IL-3, IL-6, Steel factor (SLF), and erythropoietin (EPO) (M3434; Stem Cell Technologies), then plated in culture plates, and incubated in humidified chambers at 37° C., 5% CO₂. Following 10 to 14 days of incubation, erythroid burst-forming units (BFU-E), colony forming units granulo-macrophagic (CFU-GM) and colony forming units granulocytic-erythroid-megacaryocytic-macrophagic (CFU-Mix) progenitors were scored by colony morphology. To confirm colony identity, some colonies were occasionally analyzed by Wright-Giemsa staining. CFU-pre-B were analyzed by plating the BM cells suspended in the methylcellulose media (M3630; Stem Cell Technologies) supplemented with IL-7, incubated as described above and scored for colonies after 7 days.

Results:

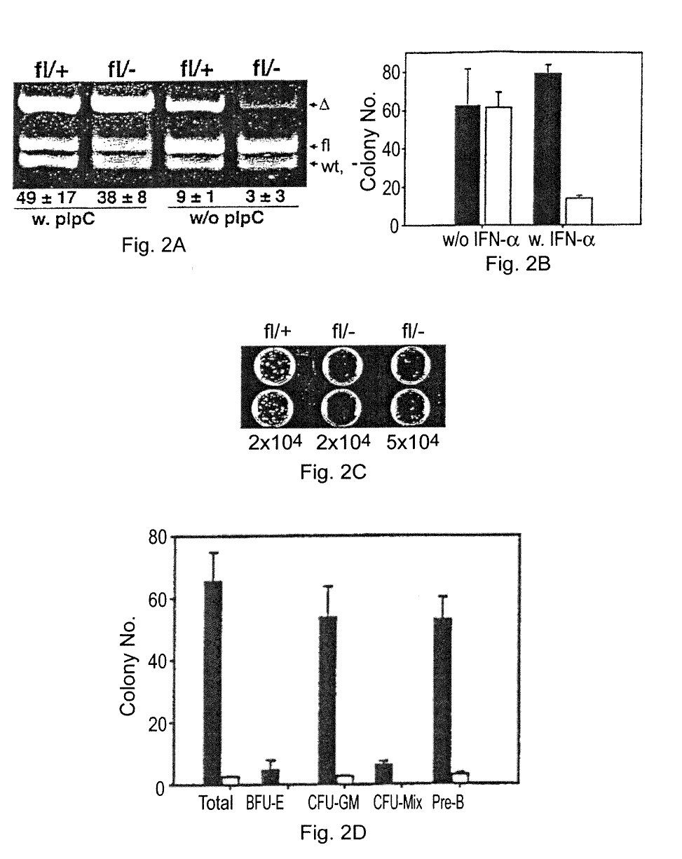

The levels of functional BM hematopoietic precursor cells, in Mx1-Cre/Casp8^{fl/-} (conditional caspase-8 knock-out) mice, which had been injected with pI-pC or interferon, decreased substantially, as determined by in vitro assays. Thus, the total number of colonies which developed in vitro from BM cells obtained from caspase-8 knock-out mice, were 7-8 fold lower than the control (Mx1-Cre/Casp8^{fl/+}) mice (FIG. 2b). Similarly, the number of myeloid and pre-B colonies developed in vitro from BM cells obtained from the caspase knock-out mice, were 15-20 fold lower than the control (Mx1-Cre/Casp8^{fl/+}) mice (FIG. 2c-d).

Clearly, the depletion of the caspase-8 gene from hematopoietic cells markedly impairs the capacity of these cells to develop in vitro.

### Example 3

### The Knock-Out of Caspase-8 in Mice Impairs the Capacity of Bone-Marrow Cells to Expand in Spleen

Materials and Methods

Animals: The conditional caspase-8 knock-out mice, Mx1-Cre/Casp8^{fl/-} and their control littermates, Mx1-Cre/Casp8^{fl/+}, were generated as described hereinabove and were injected with pI-pC 3 times to induce Cre recombinase. Female C57BL/6 mice were used as recipient mice.

In vivo colony-forming unit-spleen (CFU-S) assay: BM cells were harvested from femora of mice and the mature T cells were depleted from the BM cell preparation by MACS using anti-mouse CD4 and CD-8 microbeads (Miltenyi Biotech). Single cell suspension was then diluted to 5×10⁵ cells/ml and a 0.2 ml aliquot (1×10⁵ cells) was injected into the tail vein of an irradiated (8.5 Gy, 137 C source) 10 week-old mouse. For each experiment, 5 recipients were used for each donor genotype. Mice were administrated with antibiotics (6.7 mg/l of Ciproxin) in their drinking water. Recipients were sacrificed on 8 or 13 days after transplantation, their spleens were weighed and macroscopic colonies were counted after fixation in Bouin's solution.

Results:

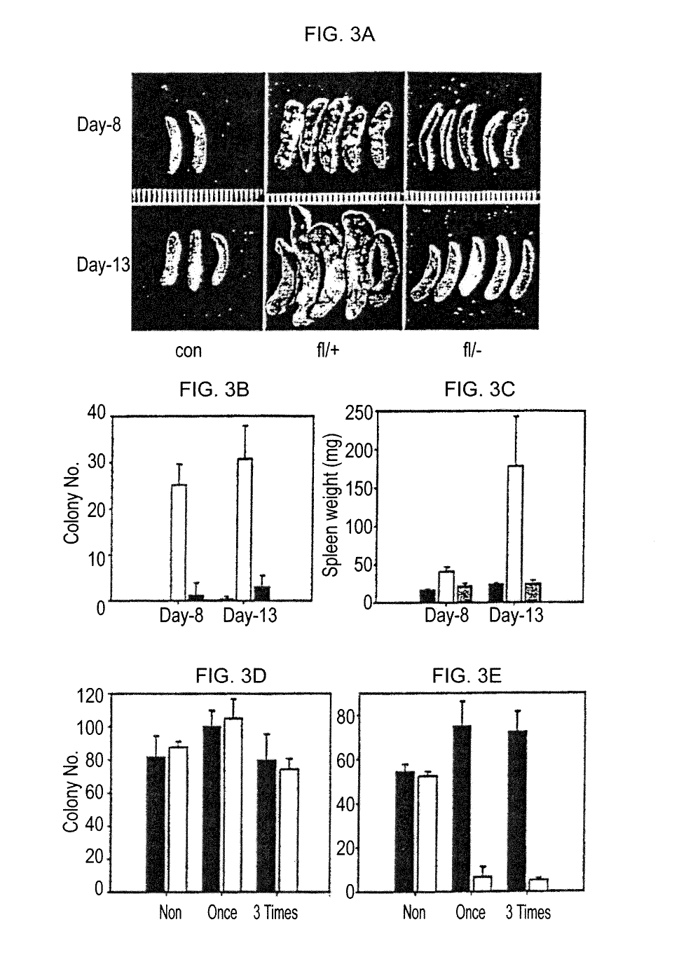

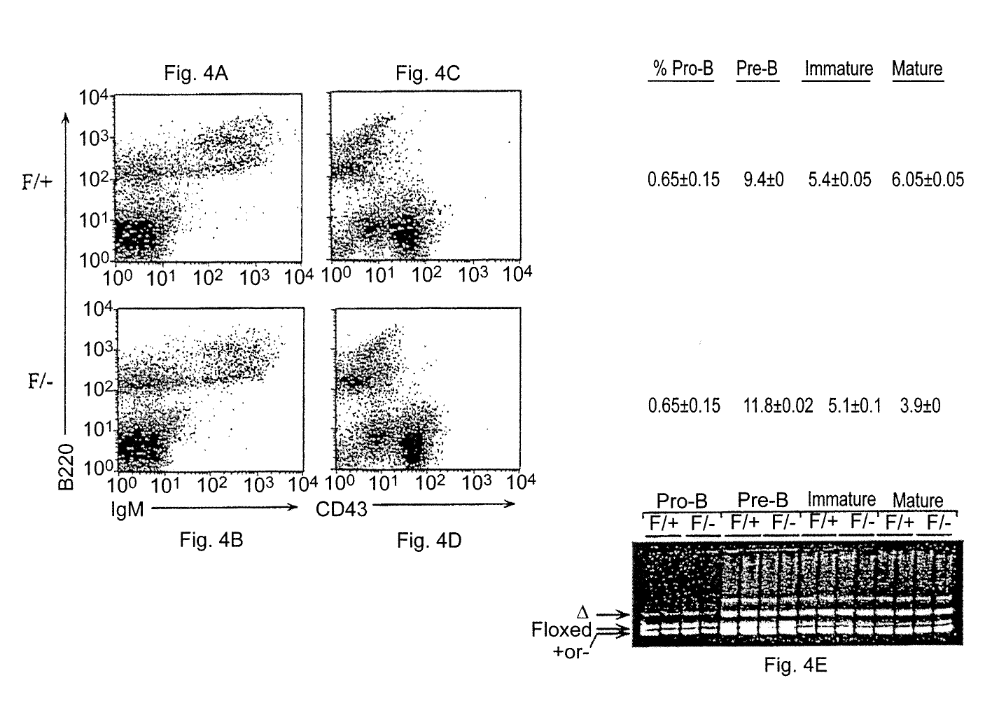

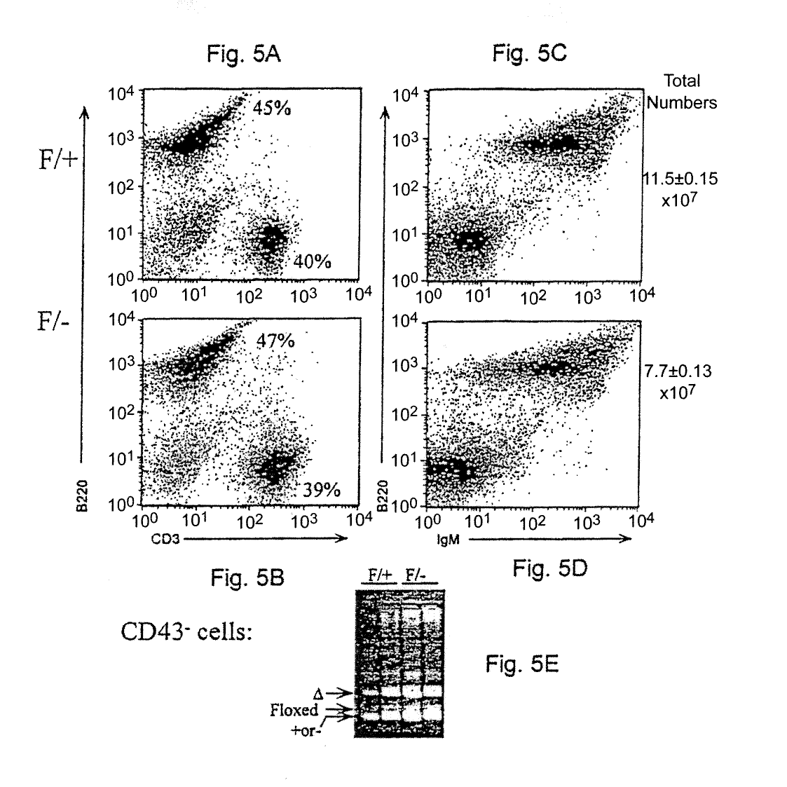

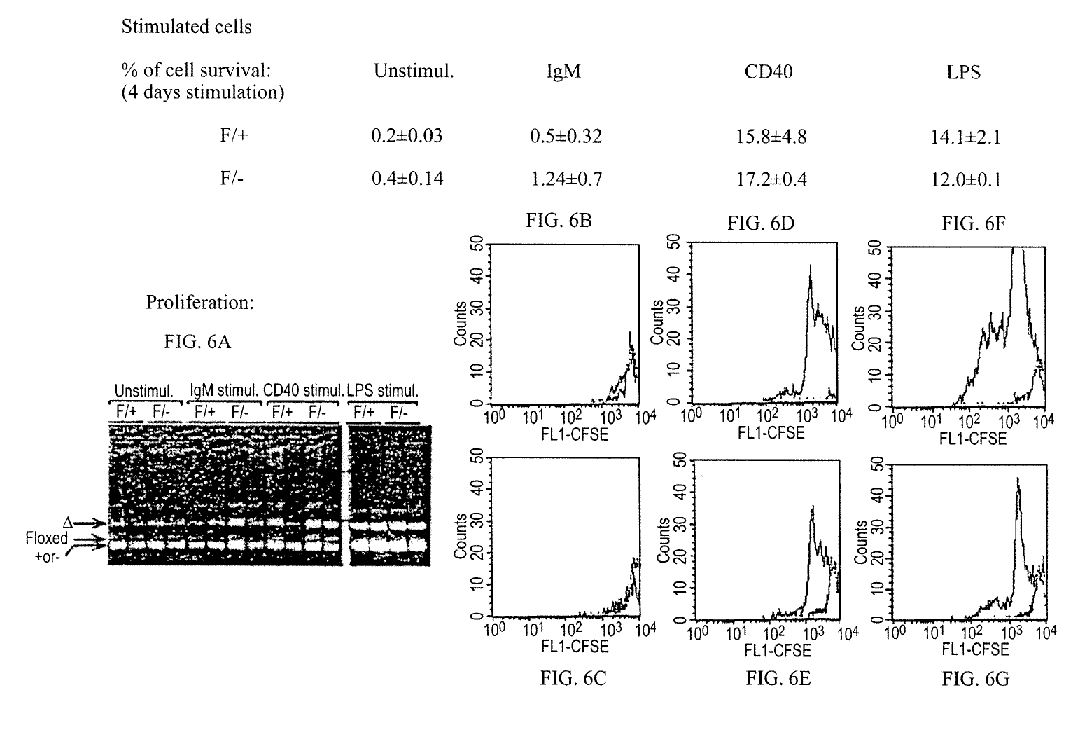

The number of hematopoietic CFUs in the spleen of irradiated mice, which had been transplanted with BM cells obtained from Mx1-Cre/Casp8^{fl/-}, decreased substantially (by approximately 90%) (FIG. 3a-c). Hence, the deletion of caspase-8 in BM cells markedly reduces the capacity of these cells to establish in spleen.

### Example 4

### The Knock-Out of Caspase-8 Impairs Transplantability of Bone-Marrow Cells

Four treatment groups of irradiation-chimera mice were generated: (i) normal (Casp8^{+/+}) mice reconstituted with Mx1-Cre/Casp8^{fl/-} BM cells; (ii) Casp8^{+/+} mice reconstituted with Mx1-Cre/Casp8^{fl/+} BM cells; (iii) Cre/Casp8^{fl/-} mice reconstituted with Casp8^{+/+} BM cells; and (iv) Cre/Casp8^{fl/+} mice reconstituted with Casp8^{+/+} BM cells. Following reconstitution, all mice were treated with pI-pC followed by the assessment of their BM hematopoietic precursor levels by in-vitro colony assay, as described in Example 2 hereinabove. The injection of pI-pC to chimera of normal (Casp8^{+/+}) mice which had been reconstituted with Mx1-Cre/Casp8^{fl/-} BM, resulted in a dramatic reduction in the number of hematopoietic CFU generated in-vitro (FIG. 3e) In contrast, no significant decrease was observed in any other treatment group of irradiation-chimera mice (FIG. 3d).

### Example 5

### The Knock-Out of Caspase-8 Arrest B Cell Stimulation

Materials and Methods:

BM cells were purified from fimura, tibia and hip bones of test animals. Erythrocytes were depleted by incubation in ACK buffer for 2 minutes. Cells were washed in cold FACS buffer (2% FCS in PBS supplemented with 0.1% sodium azide) and incubated for 5 minute at room temperature (RT) with anti-FCγ antibody to block non-specific binding of the staining antibody. Cells were stained with anti-IgM, −B220 and −CD43 antibodies for 15 minutes on ice. Cells were washed and resuspended in FACS buffer containing propidium iodide (PI) to exclude acquisition of dead cells. Cell acquisition was performed on a FACSCalibur using CellQuest software. For sorting purposes cells were manipulated as described above and in addition sorted on a FACSVantage using the CellQuest software. Following sorting cells were spun down and resuspended in 20 μl H₂O in PCR reaction tubes. The cell samples were then incubated for 10 minutes at 95° C. followed by 56° C. incubation for 1 hr with the addition of 5 μl proteinase K (PK—2 mg/ml stock). Tubes were incubated again for 10 minutes at 95° C. in order to denature the PK and a PCR mixture (Taq, primers, buffer) was added to each of the tubes as described above. Amplified DNA was separated on a 2% agarose gel to determine the level of caspase-8 deletion by CD19 Cre. Splenocytes were analysed by FACS as described above using anti-CD3, B220 and IgM antibodies. For stimulation experiments, B cells were negatively purified by MACS (Miltenye Biotech.) using CD43 beads. Cells were counted and plated in triplicates in 96 well round bottom plates in medium (DMEM supplemented with 10% FBS, Amp/Strep, Sodium pyruvate, L-Glutamine) containing 10 μg/ml IgM or 1 μg/ml CD40 or LPS 5 μg/ml in a concentration of 5×10⁵ cells/well. Four days later cell survival was determined by FACS analysis using PI and anti-B220 antibody. The rest of the cells were labelled with CFSE in PBS for 5 minutes at room temperature at a concentration of 10⁷ cells/ml. The labelling was stopped with 1 volume of 100% FBS and cells were washed twice with medium before plating them on a medium supplemented with various stimulatory compounds (described below) at a concentration of 10⁶ cells/well. Cell proliferation was analysed by FACS using PI to exclude dead cells.

Results:

Data derived from the present study indicate that caspase-8 participates in homeostasis of specific B cell subsets. Upon stimulation with LPS, B cells derived from CD19 Cre/Casp8 F/− mice exhibit arrested proliferation while control F/+ derived cells remain unaffected. This phenomenon seems to be unique in LPS stimulation as compared with CD40 or B cell receptor stimulation (IgM stimulation). This might indicate that caspase-8 has a unique role in the LPS signaling pathway, in particular in a subset of B cells most affected by LPS stimulation.

### Example 6

### The Knock-Out of Caspase-8 Inhibits Differentiation of Monocytes-Precursors

Materials and Methods:

Animals: The conditional knock-out mice Mx1-Cre/Casp8^{fl/-} (pI-pC induced deletion of floxed Casp8), and their control littermates Mx1-Cre/Casp8^{fl/+}, were generated as described in Examples 1 hereinabove. The conditional knock-out mice LysM-Cre/Casp8^{fl/-} (constitutive deletion of floxed Casp8 in myelomocytic lineage cells) and their control littermates (LysM-Cre/Casp8^{fl/+}), were generated as follows: Cre positive caspase-8 negative mice (Cre-Casp8^{+/-}) were generated by crossing Casp8 heterozygous full knockout mice (Casp8^{+/-}) with transgenic LysM-Cre mice [expressing the Cre only in cells of the myelomocytic lineage (13)]. The conditional knock-out mice LysM-Cre/Casp8^{fl/-} were generated by crossing the Casp8^{fl/fl} with the LysM-Cre/Casp8^{+/-} mice. The LysM-Cre/Casp8^{fl/+} animals which also resulted from this crossing were used as experimental control litter mates.

Cell culture: Primary cultures of bone marrow macrophages were isolated from femurs of 3-4 month old mice. The isolated BM cells were cultured with DMEM growth medium (Gibco BRL) supplemented with 20% M-FCS and 30% L929 cell-conditioned medium. Following overnight culturing, the non-adherent cells were harvested and re-suspended in fresh medium. Aliquots of 2.5×10⁵ of the cells were cultured in microwell plates and incubated for 7-10 days at 37° C., 5% CO₂, in growth medium which was replaced every 3 days. Following incubation, the adherent BM cells density was estimated by the methyl thiazole tetrazolium (MTT) test (essentially as described in J Immunol Methods. 89:271). Accordingly, culture plates were washed 3 times in PBS, then 10 μl aliquots of MTT solution (5 mg/ml) were added to each well. The plates were incubated for 4 hr, then supplemented with 100 μl of DMSO per well and analyzed for the optical density at 540 nm by a microplate reader.

Peritoneal cells were harvested by rinsing mice peritoneal cavity with sterile phosphate buffered saline (PBS, SIGMA, 10 ml/mouse) containing 2% M-FCS. The cells were then washed once with PBS, and then resuspended in RPMI-1640 medium supplemented with 10% M-FCS, glutamine, and penicillin/streptomycin. The cells were then plated on 6 cm culture dish (Nunc Inc.) at a density of 3×10⁶ cells per well, and incubated for 2-4 hr at 37° C., 5% CO2 for 2-4 hr to allow macrophage adherence.

PCR and real-time PCR verification of deletion efficiency: Genomic DNA was extracted from the adherent cultured peritoneal macrophages or bone marrow derived macrophages using a lysis buffer (10 mM Tris-HCl, pH8.5, 5 mM EDTA, 0.2% SDS, 200 mM NaCl, 0.1 mg/ml Proteinase K). and from spleen samples using a lysis buffer (10 mM Tris-HCl, pH8.5, 5 mM EDTA, 0.2% SDS, 200 mM NaCl, 0.1 mg/ml Proteinase K). The DNA was subjected to PCR analysis using the primers designated by SEQ ID NOs: 6-8 (Table 2 below). PCR amplification conditions were as followed: 5 minutes at 95° C. followed by 33 cycles, each consisting of 30 sec at 94° C., 30 sec at 58° C. at 30 sec at 72° C., with a final cycle of 10 minutes at 72° C.

For more quantitative evaluation, the extent of deletion was assessed by real-time PCR. The assay was performed in a reaction volume of 20 μl containing 10 ng DNA, 10 pmole of oligonucleotide primers, 50 pmole of oligonucleotide probes and 10 μl of Taqman 2×PCR master mix (Applied Biosystems). The PCR reaction (40 cycles of: 95° C. for 15 seconds, 60° C. for 60 seconds) was performed using the ABI Prism 7000 Sequence Detection System (PE Applied Biosystems). The caspase-8 oligonucleotide primers utilized are shown in Table 2 (SEQ ID NOs: 9-10).

Caspase-8 gene levels were normalized by quantifying the NIK gene present in the same DNA samples. NIK gene quantification was effected using the NIK primers shown in Table 2 (SEQ ID NOs: 11-12) and PCR amplification conditions similar to those used for caspase-8 quantification.

TABLE 2
Oligonucleotide primers and probes
used for PCR and real time PCR

| | | | |
|---|---|---|---|
| Primer | | SEQ ID | |
| Sequences | Sequence | NO: | Comments |

| | | | |
|---|---|---|---|
| 22709 | tagcctctttggggttgttctactg | 6 | Sense for |
| | | | caspase-8, |
| | | | flox, and |
| | | | deleted allele. |
| 16402 | cgcggtcgacttatcaagaggtag | 7 | Antisense for |
| | aagagctgtaac | | flox and |
| | | | caspase-8 |
| FloC4 | gcgaacacgccgtgtttcaagggc | 8 | Antisense for |
| | | | deleted allele |
| Realtime | ggaaacaagctggtagctgaca | 9 | Sense for |
| PCR sense | | | caspase-8 |
| Realtime | cctgggtcaacacaagatgct | 10 | Antisense for |
| PCR | | | caspase-8 |
| antisense | | | |
| Nik primer | agcctcctctaccgccagaa | 11 | Sense for NIK |
| Nik primer | gtgccagactcctccttgct | 12 | Antisense for |
| | | | NIK |
| Casprobe | 6-FAM (6-caroxy-fluorescein)- | 13 | Probe for |
| | ttaacttcctcacttgatcat- | | caspase-8 |
| | MGB(minor grove binder) | | |
| Nik probe | 6-FAM-accagaaccgagcaaa-MGB | 14 | Probe for |
| | | | NIK |

Three oligonucleotides were used for real-time PCR: a sense, an antisense and a probe. All of them are specific for the target gene (caspase-8 and NIK) and are able to bind it. The probe is an oligonucleotide with a reporter dye at the 5′ end and a quencher dye at the 3′ end. The fluorescent reporter dye (FAM) is attached covalently to the 5′ end and the reporter is quenched by MGB, bound to the 3′ end. When the probes is intact, the quencher dye absorbs the fluorescence of the reporter dye, and fluorescence emission does not occur. By the 5′-exonuclease activity of the Taq polymerase the probe is hydrolyzed and the reporter dye is separated from the quencher, resulting in an increase in fluorescence emission. During PCR amplification, if the target of interest is present, the probe specifically anneals to the target. The Taq polymerase cleaves the probe, allowing an increase in fluorescence emission. This increase in fluorescence is measured cycle by cycle and is a direct consequence of the amplification process.

The threshold cycles (Ct) of the reaction were calculated from the ΔRn (ΔΔRn=Rn⁺−Rn⁻, where Rn⁺ is the fluorescence emission of the product at the each time point and Rn⁻ is the fluorescence emission of base line) versus cycle number plot and variations in the caspase-8 gene levels were compared to those of NIK using the following calculation: Fold change=2^{-ΔΔCt}, where ΔΔCt=(Ct_{casp8}−Ct_{NIK})_{sample DNA}−(Ct_{casp8}−Ct_{NIK})_{non-deleted control DNA}.

The % deletion of caspase-8 gene of each sample was deducted from the 2^{-ΔΔCt} values using the following calculation: % deletion=(1−2^{-ΔΔCt})×200.

Results:

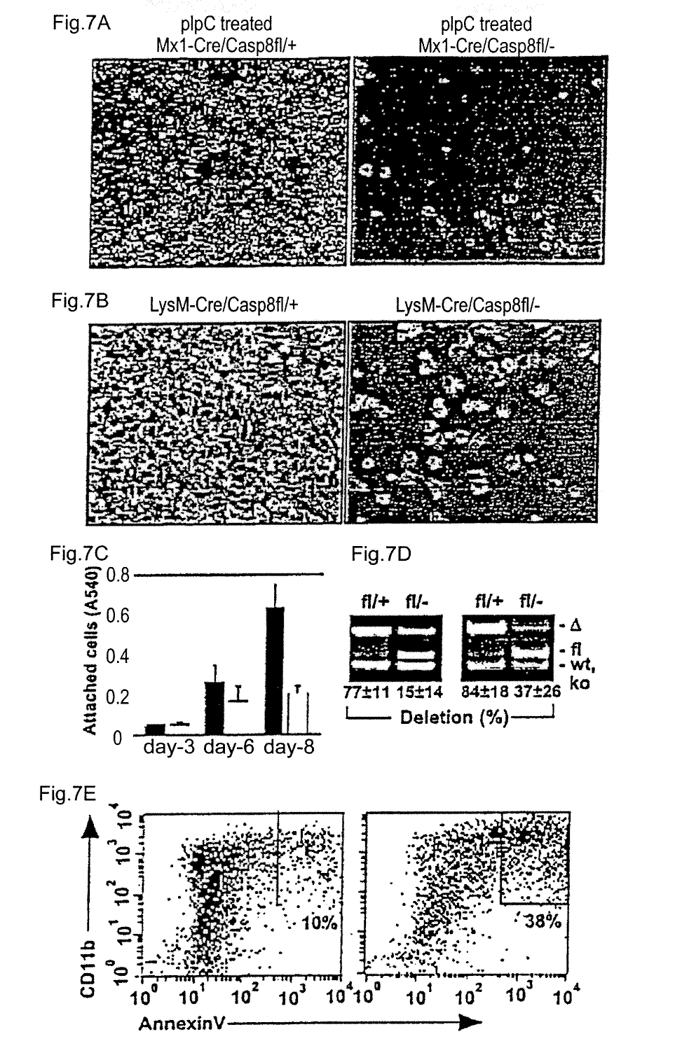

BM cells were isolated from conditional knock-out mice (Mx1-Cre/Casp8^{fl/-}) and from the control (Mx1-Cre/Casp8^{fl/+}) mice which had been injected with pIpC. The cells were cultured in the presence of M-CSF to stimulate differentiation of monocyte precursors into macrophages. Consequently, substantially fewer cells adhered and attained macrophage morphology in the Mx1-Cre/Casp8^{fl/-} culture, as compared with the Mx1-Cre/Casp8^{fl/+} culture (FIG. 7a), thus indicating that caspase-8 is required for the M-CSF induced macrophage differentiation in vitro.

BM cells isolated from the conditional knock-out mice LysM-Cre/Casp8^{fl/-} mice, and from their control littermates LysM-Cre/Casp8^{fl/-}, were cultured in growth medium which had not been supplemented with M-CSF and comparatively analyzed for hematopoietic colony formation, using the procedure described in Example 2 hereinabove. The resulting density of myeloid CFU in the culture which derived from the knock-out mice, did not significantly differ from the CFU density in the culture derived from the control mice (data not shown), thus indicating that myeloid precursors function in the knock-out mice was normal. However, when M-CFS was provided to the cell cultures to stimulate in vitro differentiation, substantially fewer cells differentiated into macrophages in the LysM-Cre/Casp8^{fl/-} derived culture, as compared with the control culture (FIGS. 7b-c).

Adhering macrophages sampled from the culture of BM cells derived from LysM-Cre/Casp8^{fl/-} mice were analyzed by PCR. The PCR analysis revealed that the foxed caspase-8 alleles had not been deleted from these cells (FIG. 7d).

Culture staining with the cell death marker Annexin-V resulted in a substantially higher density of positively stained non-adherent monocytic cells in the LysM-Cre/Casp8^{fl/-} derived culture, than that observed in the LysM-Cre/Casp8^{fl/+} derived culture (FIG. 7e). These findings indicate that in vitro differentiation of monocytes-precursors into macrophages requires caspase-8 expression and that cells which do not express this enzyme die.

Macrophages derived from peritoneal exudates (PEC) of the LysM-Cre/Casp8^{fl/+} and LysM-Cre/Casp8^{fl/-} mice exhibited approximately 90% and 50% deletion of the floxed allele, respectively (FIG. 7f). These findings indicate that in vivo differentiation of monocytes-precursors into macrophages requires caspase-8 expression and that abolishment of caspase-8 expression compromises the growth and/or survival of macrophages.

Hence the findings described above clearly indicate that the knock-out of caspase-8 in mice inhibits monocytes-precursors differentiation to macrophages.

### Example 7

### The Knock-Out of Caspase-8 Impairs Embryonic Hematopoiesis in Mice

Materials and Methods:

Animals: The conditional knock-out mice Tie1-Cre/Casp8^{fl/-} (constitutive deletion of floxed Casp8 in endothelial cells from embryonic day 8; promoter active in 13% of the hematopoietic lineage cells) and their control littermates (Tie1-Cre/Casp8^{fl/+}), were generated as follows: Cre positive caspase-8 negative mice (Cre-Casp8^{+/-}) were generated by crossing Casp8 heterozygous full knockout mice (Casp8^{+/-}) with transgenic Tie1-Cre mice [expressing the Cre only in endothelial cells; Gustafsson et al., J. Cell Sci. 114:671-676, 2001]. The conditional knock-out mice Tie1-Cre/Casp8^{fl/-} were generated by crossing the Casp8^{fl/fl} with the Tie1-Cre/Casp8^{+/-} mice. The Tie1-Cre/Casp8^{fl/+} animals which also resulted from this crossing were used as experimental control litter mates.

In-vitro hematopoietic colony assay: E10.5 Yolk-sacs and total embryos were dissected, mechanically disrupted, and filtered through 15 mm nylon mesh. Cell viability was determined by trypan blue staining. Samples of 5×10⁴ viable cells were plated in RPMI medium containing methylcellulose and cytokines, incubated in 37° C. and scored 7 days later.

Results:

Tie-1 Cre transgenic mice constitutively express Cre in endothelial cells at early embryonic stage. Crossing the casp8^{flox/flox} mice with a transgenic mouse line expressing Cre recombinase under the control of Tie1 promoter resulted in embryonic lethality of Tie1-Cre/casp8^{fl/-} mice. The mice died at embryonic-day 10.5-11.5 and exhibited impaired heart muscle development, congested accumulation of erythrocytes and underdeveloped yolk-sac vasculature, all of which indicated that the caspase-8 knockout mice died due to endothelial cell defect. Analysis of E11.5 Tie1-Cre/casp8^{fl/-} embryos and yolk-sacs primary vessels network using whole-mount immunohistochemistry PECAM (CD-31 endothelial specific antibody) staining, revealed that yolk-sac remodeling was defective in these animals.

No hematopoietic colony-forming units (CFU) could be recovered from Casp8^{-/-} mice (caspase-8 knock-out), while the hematopoietic CFU which could be recovered from the conditional knock-out Tie1-Cre/casp8^{fl/-}, but in fewer CFU as compared to the control Tie1-Cre/casp8^{fl/+} (Table 3).

TABLE 3
In vitro embryonic hematopoietic colony assay

| Genotype | Casp8+/− | Casp8−/− | Tie1Cre/Casp8^{fl/+} | Tie1Cre/Casp8^{fl/-} |
|---|---|---|---|---|
| Colony | 50-150 | 0-5 | 50-100 | 30-60 |
| number | | | | |

The reduced colony number in embryonic hematopoietic CPU in the Tie1Cre/Casp8^{fl/-} mice indicates that hematopoiesis was impaired in embryos of Tie-1 caspase-8 knock-out mice further substantiating the role of caspase-8 in the hematopoietic process.

In conclusion, the results described hereinabove clearly indicate that caspase-8 serves a critical role in regulating hematopoiesis and thus can be utilized as a target for treating hematopoietic disorders, and in particular disorders which are characterized by hyperproliferation of hematopoietic cells, such as leukemia.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

In conclusion, the results described hereinabove clearly indicate that caspase-8 serves a critical role in regulating hematopoiesis and thus can be utilized as a target for treating hematopoietic disorders, and in particular disorders which are characterized by hyperproliferation of hematopoietic cells, such as leukemia.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED BY NUMERALS

### Additional References are Cited Hereinabove

1. Shi, Y., Mechanisms of caspase activation and inhibition during apoptosis. Mol Cell, 2002. 9(3): p. 459-70.
2. Alam, A., et al., Early activation of caspases during T lymphocyte stimulation results in selective substrate cleavage in nonapoptotic cells. J Exp Med, 1999. 190(12): p. 1879-90.
3. Boissonnas, A., et al., Differential requirement of caspases during naive T cell proliferation. Eur J Immunol, 2002. 32(10): p. 3007-15.
4. De Maria, R., et al., Negative regulation of erythropoiesis by caspase-mediated cleavage of GATA-1. Nature, 1999. 401(6752): p. 489-93.
5. Kennedy, N. J., et al., Caspase activation is required for T cell proliferation. J Exp Med, 1999. 190(12): p. 1891-6.
6. Faouzi, S., et al., Anti-Fas induces hepatic chemokines and promotes inflammation by an NF-kappa B-independent, caspase-3-dependent pathway. J Biol Chem, 2001. 276(52): p. 49077-82.
7. Chaudhary, P. M., et al., Activation of the c-Jun N-terminal kinase/stress-activated protein kinase pathway by overexpression of caspase-8 and its homologs. J Biol Chem, 1999. 274(27): p. 19211-9.
8. Coletti, D., et al., TNFalpha inhibits skeletal myogenesis through a PW1-dependent pathway by recruitment of caspase pathways. Embo J, 2002. 21(4): p. 631-42.
9. Boldin, M. P., et al., Involvement of MACH, a novel MORT1/FADD-interacting protease, in Fas/APO-1- and TNF receptor-induced cell death. Cell, 1996. 85(6): p. 803-15.
10. Muzio, M., et al., FLICE, a novel FADD-homologous ICE/CED-3-like protease, is recruited to the CD95 (Fas/APO-1) death-inducing signaling complex. Cell, 1996. 85(6): p. 817-27.
11. Varfolomeev, E. E., et al., Targeted disruption of the mouse Caspase-8 gene ablates cell death induction by the TNF receptors, Fas/Apo1, and DR3 and is lethal prenatally. Immunity, 1998. 9(2): p. 267-76.
12. Kuhn, R., et al., Inducible gene targeting in mice. Science, 1995. 269(5229): p. 1427-9.
13. Clausen, B. E., et al., Conditional gene targeting in macrophages and granulocytes using LysMcre mice. Transgenic Res, 1999. 8(4): p. 265-77.
14. Scaffidi, C., et al., Two CD95 (APO-1/Fas) signaling pathways. Embo J, 1998. 17(6): p. 1675-87.
15. Yin, X. M., et al., Bid-deficient mice are resistant to Fas-induced hepatocellular apoptosis. Nature, 1999. 400(6747): p. 886-91.
16. Schuchmann, M., et al., Dominant negative MORT1/FADD rescues mice from CD95 and TNF-induced liver failure. Hepatology, 2003. 37(1): p. 129-35.
17. Kim, S. O., K. Ono, and J. Han, Apoptosis by pan-caspase inhibitors in lipopolysaccharide-activated macrophages. Am J Physiol Lung Cell Mol Physiol, 2001. 281(5): p. L1095-105.
18. Sordet, O., et al., Specific involvement of caspases in the differentiation of monocytes into macrophages. Blood, 2002. 100(13): p. 4446-53.
19. Salmena, L., et al., Essential role for caspase-8 in T-cell homeostasis and T-cell-mediated immunity. Genes Dev, 2003. 17(7): p. 883-95.
20. Sakamaki, K., et al., Ex vivo whole-embryo culture of caspase-8-deficient embryos normalize their aberrant phenotypes in the developing neural tube and heart. Cell Death Differ, 2002. 9(11): p. 1196-206.
21. Zhang, J., et al., Fas-mediated apoptosis and activation-induced T-cell proliferation are defective in mice lacking FADD/Mort1. Nature, 1998. 392(6673): p. 296-300.
22. Kabra, N. H., et al., T cell-specific FADD-deficient mice: FADD is required for early T cell development. Proc Natl Acad Sci USA, 2001. 98(11): p. 6307-12.
23. Walsh, C. M., et al., A role for FADD in T cell activation and development. Immunity, 1998. 8(4): p. 439-49.
24. Newton, K., et al., A dominant interfering mutant of FADD/MORT1 enhances deletion of autoreactive thymocytes and inhibits proliferation of mature T lymphocytes. Embo J, 1998. 17(3): p. 706-18.
25. Zornig, M., A. O. Hueber, and G. Evan, p53-dependent impairment of T-cell proliferation in FADD dominant-negative transgenic mice. Curr Biol, 1998. 8(8): p. 467-70.
26. Segura, I., A et al., Inhibition of programmed cell death impairs in vitro vascular-like structure formation and reduces in vivo angiogenesis. FASEB J., 2002 16: p. 833-841

1. (canceled)

2. A method of inhibiting hematopoiesis in a subject comprising downregulating an expression or activity of caspase-8 in the subject, wherein downregulating the expression or activity of caspase-8 is effected by: (a) an antibody, an antibody fragment, z-VAD-fmk, IEDT-fmk, or DEVD-fmk which binds caspase-8; (b) an enzyme which cleaves caspase-8; (c) an antisense polynucleotide capable of specifically hybridizing with an mRNA transcript encoding caspase-8; (d) a ribozyme which specifically cleaves transcripts encoding caspase-8; (e) a small interfering RNA (siRNA) molecule which specifically cleaves caspase-8 transcripts; (f) a non-functional analogue of at least a catalytic or binding portion of caspase-8; (g) a vector for inducing and/or enhancing the endogenous production of an endogenous inhibitor of caspase-8; and/or (h) a vector for inhibiting the endogenous production of endogenous caspase-8.

3. The method of claim 2, wherein downregulating the expression or activity of caspase-8 is effected by the antisense polynucleotide.

4. (canceled)

5. The method of claim 2, wherein downregulating the expression or activity is effected by the antibody or the antibody fragment.

6. (canceled)

7. The method of claim 2, wherein downregulating the expression or activity of caspase-8 is effected by the small interfering RNA (siRNA) molecule.

8.-62. (canceled)

63. The method of claim 3, wherein the antisense polynucleotide comprises the sequence set forth in SEQ ID NO: 16.

64. The method of claim 5, wherein the antibody fragment is a Fab or a ScFv fragment.

65. The method of claim 5, wherein the antibody or antibody fragment blocks association of caspase-8 with Fas Associated protein with Death Domain.

66. The method of claim 5, wherein the antibody or antibody fragment is a monoclonal antibody or monoclonal antibody fragment.

67. The method of claim 5, wherein the antibody or antibody fragment is a humanized antibody or humanized antibody fragment.

68. The method of claim 7, wherein the small interfering RNA (siRNA) molecule comprises the sequence set forth in SEQ ID NO: 15.

69. The method of claim 2, wherein the method comprises downregulating an expression or activity of an α1 splice variant or an α2 splice variant of caspase-8.

70. The method of claim 2, wherein the vector comprises a viral vector.

71. The method of claim 2, wherein one or more of (a)-(g) is delivered to the subject by direct injection into bone marrow.
